(19) United States
(12) Patent Application Publication
(10) Pub. No.: US 2008/0234718 A1
(43) Pub. Date: Sep. 25, 2008

(54) Ballasted Intragastric Balloon, Use Of An Absorbing Body And/Or Heavy Solid Bodies To Form A Ballast Inside Such A Balloon

(76) Inventors: Pascal Paganon, Serezin du Rhone (FR); Jean-Paul Gilbert Ricol, Saint Germain sur L'Arbresle (FR)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(21) Appl. No.: 10/595,505

(22) PCT Filed: Oct. 15, 2004
(86) PCT No.: PCT/FR2004/002650

§ 371 Date: May 15, 2008

(30) Foreign Application Priority Data
Oct. 23, 2003 (FR) 0312426

(51) Int. Cl. A61M 29/02

(52) U.S. Cl. 606/192; 606/191

(57) ABSTRACT

Please cancel the previous Abstract and insert the following new Abstract. The invention relates to an expandable intragastric balloon, for implantation into the stomach of a patient, to reduce the volume thereof for the treatment of obesity. Said balloon is provided with at least one first flexible pouch, capable of shifting from a folded position to an expanded position by introducing an inflation fluid into the first pouch, said expanded position giving a functional form to the balloon. Said balloon is characterized in that it comprises a ballasting means, for making said balloon substantially heavy to improve the positioning thereof in the stomach.

### PRIORITY CLAIM

This patent application is a U.S. National Phase of International Application No. PCT/FR2004/002650, having an International Filing Date of Oct. 15, 2004, which claims priority to French Patent Application No. FR 0312426, having a Filing Date of Oct. 23, 2003, the disclosures of which are incorporated herein by reference in their entirety.

### FIELD OF THE INVENTION

This invention relates to the technical field of implantable devices intended to be used for the treatment of obesity, in particular morbid obesity, and very specifically to implants capable of artificially reducing the volume of the stomach, in particular in order to produce a sensation of satiety in the patient.

This invention relates to an expandable intragastric balloon intended to be implanted inside the stomach of a patient so as to reduce the volume of the stomach for the treatment of obesity, which balloon is equipped with at least one first flexible pouch capable of changing from a folded position to an expanded position by introducing an inflation fluid inside the first pouch, wherein the expanded position gives the balloon its functional form.

The invention relates to a new use of absorbent bodies in the medical field.

The invention also relates to a new use of dense solid bodies in the medical field.

### BACKGROUND OF THE INVENTION

It is known to implant foreign bodies, in particular intragastric balloons, in the stomach of patients suffering from obesity, so as to generate in these patients a rapid sensation of satiety.

The foreign bodies thus implanted generally have a predetermined volume enabling the space of the stomach reserved for food to be reduced, while reducing the speed at which the food passes into the stomach.

The intragastric balloons used in this context generally comprise a flexible pouch capable of changing from a folded position to an expanded position when an inflation fluid is introduced into the pouch. Thus, the pouch is flexible and generally equipped with an aperture closed off by a valve through which the inflation can be achieved by an inflation fluid, and in particular a gas.

The balloon is generally inserted orally, in its folded form, so as to facilitate its insertion through the oesophagus.

Once positioned in the stomach, the balloon can be inflated until it reaches the expanded position in which it occupies a given volume inside the stomach.

Intragastric balloons inflated by a gas generally provide good results in the treatment of obesity, but nevertheless have a number of disadvantages.

In particular, balloons inflated with air are generally rather poorly positioned in the gastric cavity.

They indeed have a tendency to move into the upper portion of the stomach, and to be positioned at the level of the cardia, thus hindering the passage of the food into the stomach.

However, even if they are actually intended to reduce the speed at which food passes into the stomach, intragastric balloons must not fully impede the functioning of the digestive system.

In addition, improper positioning of the balloon in the stomach can be a significant source of discomfort for the patient.

The positioning of the intragastric balloon in the stomach therefore appears to be an essential parameter, the control of which makes it possible to reduce the adverse effects associated with specific technique and to improve the efficacy of the treatment.

### SUMMARY OF THE INVENTION

The features of the present invention are therefore to overcome the various disadvantages listed above and to propose a new expandable intragastric balloon for the treatment of obesity, of which the structure makes it possible to improve the positioning of the balloon in the patient's stomach, and to limit the adverse effects of the implant on the functioning of the digestive system while ensuring rapid and easy implantation.

Another feature of the invention is to propose a new intragastric balloon that can be produced simply and inexpensively, and that is effective in the treatment of obesity.

Another feature of the invention is to propose a new intragastric balloon that can easily be manipulated, and of which the placement requires only a limited number of operations by the surgeon.

Another feature of the invention is to propose a new intragastric balloon that is well supported by the patient.

Another feature of the invention is to propose a new intragastric balloon of which the safety is improved.

Another feature of the invention is to propose a new intragastric balloon that is particularly strong and that enables the inflation fluid losses to be limited, and of which the duration of effectiveness is increased.

The invention also aims to propose a new use of an absorbent body in the medical field.

The features of the invention are achieved by an expandable intragastric balloon intended to be implanted inside the stomach of a patient so as to reduce the volume of the stomach for the treatment of obesity, wherein said balloon is equipped with at least one first flexible pouch capable of changing from a folded position to an expanded position by the introduction of an inflation fluid inside the first pouch, which expanded position gives the balloon its functional form, characterized in that it comprises means for ballasting said balloon enabling the balloon to be substantially weighted so as to improve its positioning in the stomach.

The features of the invention are also achieved by the use of an absorbent body to form a ballast for an expandable intragastric balloon.

The features of the invention are also achieved by the use of dense solid bodies to form a ballast for an expandable intragastric balloon.

### BRIEF DESCRIPTION OF THE DRAWINGS

Other features and advantages of the invention will become more clear from the following description, and with the help of the appended drawings, given purely by way of illustrative and non-limiting examples, wherein:

FIGS. 1 to 3 show, in a diametral cross-section view, three alternative embodiments of an intragastric balloon according to the invention in its maximum expansion position, provided with ballasting means.

FIG. 4 shows, in a side view, an alternative embodiment of an intragastric balloon provided with ballasting means.

FIG. 5 shows, in a side cross-section view, another alternative embodiment of an intragastric balloon according to the invention and provided with ballasting means.

FIG. 6 shows, in a side cross-section view, another alternative embodiment of the intragastric balloon according to the invention and provided with ballasting means.

### DESCRIPTION OF THE INVENTION

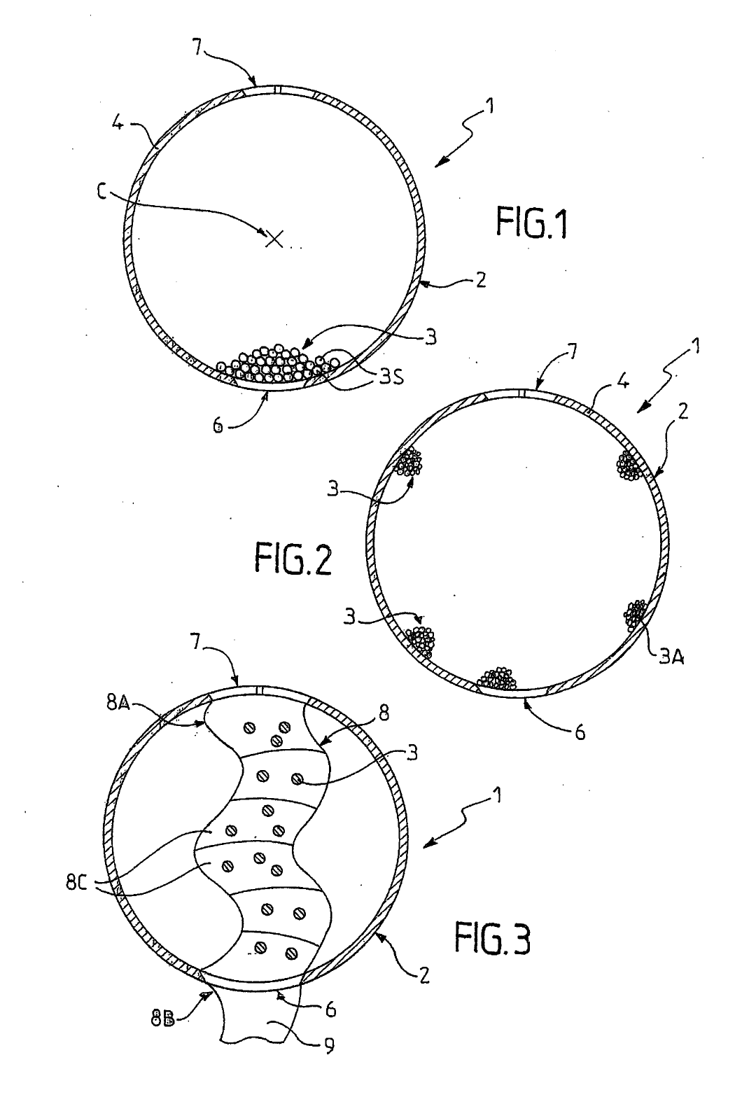

FIG. 1 shows an intragastric balloon 1 according to the invention. Such a balloon is intended to be used to treat obesity and is designed to be implanted in the stomach of a patient so as to reduce the volume of the gastric cavity by occupying a predetermined volume thereof.

The use of such a balloon thus makes it possible to reduce the space available for food and to produce a rapid sensation of satiety in the patient.

The intragastric balloon 1 according to the invention is expandable, i.e. it can have a folded configuration (not shown in the Figs.) in which it occupies a reduced volume facilitating the implantation of the balloon, an din particular its passage through the oesophagus, and an expanded configuration, corresponding to a predetermined volume giving the balloon its functional form (FIG. 1).

It is thus in this expanded form that the balloon will be capable of being used to treat the patient's obesity.

In its folded form, the intragastric balloon 1 is preferably in the form of a cigar in order to be introduced more easily into the oesophagus. The balloon 1 can also, in its folded position, be compressed by a support cover capable of further facilitating the implantation of the balloon.

According to the invention, the intragastric balloon 1 is equipped with at least one first pouch 2, preferably flexible, capable of changing from a folded position to an expanded position when an inflation fluid, for example a gas, is introduced into said pouch 2.

The first pouch 2 preferably forms the external envelope of the intragastric balloon 1, but it can also of course be contained in a separate envelope, such as a second flexible pouch 5 (FIG. 5).

The intragastric balloon 1, and in particular the pouch 2, are preferably made of flexible materials, for example elastomers such as silicone.

The inflation fluid used is preferably air that the surgeon introduces into the first pouch 2, when the intragastric balloon 1 is properly positioned in the patient's stomach.

To this end, the first pouch 2 preferably comprises a valve 7, for example a one-way valve, which valve 7 is connected to an inflation system so as to enable the intragastric balloon 1 to be filled.

To prevent leakage of the inflation fluid through the surface of the first pouch 2, the latter is preferably coated, at least partially, with an impermeable parylene coating. This measure is all the more necessary when the inflation fluid is a gas.

According to the invention, and as shown in FIGS. 1 to 5, the intragastric balloon 1 according to the invention comprises ballasting means 3 enabling the balloon to be substantially weighted so as to improve its positioning in the stomach.

The ballasting means 3 are preferably structurally integrated with the balloon, i.e. the intragastric balloon 1 according to the invention already comprises, before its implantation into the stomach, all of the physical and functional components of the ballasting means 3, so that the latter is an integral part of the balloon structure.

It is precisely this particular structure that gives the intragastric balloon 1 according to the invention its properties and advantages over the prior art.

Thus, the ballasted intragastric balloon 1 according to the invention is positioned in the upper portion of the stomach, but at an adequate distance from the cardia so as not to inhibit the passage of food into the stomach.

In addition, the intragastric balloon 1 according to the invention, due to its structure and moderate weight, has good mobility in the stomach, which mobility is particularly useful and necessary during digestion phases.

According to an alternative of the invention, the ballasting means 3 are arranged inside said first pouch 2 (FIG. 1).

Obviously, the ballasting means 3 can also be arranged in other locations, for example in the wall 4 forming the surface of said first pouch 2, in particular in recesses (not shown in the figures) formed inside said wall 4.

According to an alternative of the invention, the ballasting means 3 comprise at least one solid dense body 3S capable of forming a ballast.

By way of a non-limiting example, the solid body 3S can be formed by a single ball made of a dense material, so as to reduce the volume required to obtain the desired result.

Thus, the volume occupied by the intragastric balloon 1 equipped with its ballasting means 3 in the folded configuration must be as small as possible so as to facilitate the introduction or extraction of the balloon through the patient's oral passageways, for example the oesophagus.

The ballasting means 3 advantageously comprise a plurality of solid dense bodies 3S.

The ballasting means 3, thus divided, facilitates the introduction or extraction of the balloon through the oral passageways.

The ballasting means 3 are thus preferably formed by a plurality of small balls, made, for example, of tungsten, which is preferred for its biocompatible nature, or stainless steel of which the surface has been treated against acid degradation.

It is particularly advantageous for the solid dense bodies 3S to be preferably connected to one another so as to limit their relative mobility.

Thus, the connection of the solid bodies to one another enables them to limit the risk of shocks or noises capable of adversely affecting the patient's comfort.

The solid bodies 3S can be connected by magnetic connections, or by flexible or rigid connections such as wire parts not shown in the figures.

The ballasting means 3 preferably also comprises spaces (not shown) placed between two consecutive solid bodies 3S so as to prevent shocks. The spacers are preferably made of an elastomer material, such as silicone.

According to an embodiment not shown in the figures, one of the ends of the wire connecting the solid bodies 3S is secured to the valve 7, which enables the mobility of the ballasting means 3 to be limited in the intragastric balloon 1. The valve 7 then advantageously forms means for attachment and support for the ballasting means 3.

According to another alternative of the invention, the ballasting means 3 include at least one absorbent body 3A capable of forming a ballast in the presence of moisture.

The absorbent body 3A is advantageously arranged inside the first pouch 2 so that the surgeon, after having inserted the balloon 1 into the stomach, only has to add a predetermined amount of liquid, and, for example, water or physiological liquid, into said first pouch 2, so as to make the ballasting means 3 functional, as the absorbent body 3A soaks up the liquid.

According to a first alternative of the invention, the absorbent body 3A is formed by a foam or a sponge, and preferably a sponge made of a polyvinyl alcohol-based material.

Such a sponge 3A has the advantage of being highly compressible, which facilitates the production of the intragastric sponge 1, in particular the introduction of the sponge 3A in the compressed position into the first pouch 2 through a sealable aperture, and the implantation of the balloon 1 and in particular its passage through the oesophagus, as the sponge 3A thus compressed has reduced bulk.

After implantation of the balloon 1, the surgeon can insert into the first pouch 2, a liquid, for example water, capable of being absorbed by the sponge 3A. He can then introduce the inflation liquid, for example, air, so as to give the balloon its functional form and enable the sponge 3A to change from a compressed position in which it occupies a reduced volume, to an expanded position. These steps can be reversed.

In a particularly advantageous manner, the sponge 3A, in its expanded position, substantially occupies the entire internal volume of the first pouch 2 (FIG. 6). Some of the cells of the sponge 3A are then filled with liquid, while others are filled with the inflation fluid.

Obviously, it is also possible to consider producing a sponge 3A that occupies only a portion of the internal volume of the first pouch 2.

According to an alternative not shown in the figures, the sponge 3A can also be contained in another pouch, for example made of silicone, placed inside the first pouch 2. In this case, the inflation fluid is preferably introduced into the first pouch 2, while the liquid is introduced into the other pouch, so as to be absorbed by the sponge 3A. Means for connection with a liquid container, for example a valve 7, must then be provided on the pouch containing the sponge 3A, so as to enable the liquid to be introduced.

According to another alternative of the invention, the ballasting means 3 include a plurality of absorbent bodies 3A, as such a division of the ballasting means 3 facilitates the insertion of the balloon and its passage through the patient's oral passageways.

The absorbent bodies 3A are then preferably formed by super-absorbent particles, such as water grains. Such particles are indeed capable of absorbing up to 200 times their weight in water.

The super-absorbent particles are preferably small, with a particle size preferably ranging from 100 μm to around 1 mm.

Obviously, it is possible to consider using larger particles, for example larger than one millimetre, without going beyond the scope of the invention.

The size of the particles forming the absorbent bodies 3A can also be smaller than 100 μm, but it is preferable to avoid excessively small particle sizes which are capable of causing problems of pollution if the production of the intragastric balloon 1 requires working in the clean room.

In the dry state, these super-absorbent particles are in divided form, but they have the property of become mutually agglomerated in the presence of a liquid, or, more generally, moisture (FIG. 2).

In the presence of moisture, these super-absorbent particles then form “packets or agglomerates” capable of adhering to the wall 4 of the first pouch 2 (FIG. 2).

These super-absorbent particles thus improve the distribution of weight of the ballasting means 3 within the intragastric balloon 1, by comparison with the solid dense bodies 3S shown in FIG. 1, which have a tendency to be positioned in the bottom of the intragastric balloon 1.

In a particularly advantageous manner, the super-absorbent particles or water grains are capable of dividing again, which significantly facilitates the contraction of the intragastric balloon 1 and its passage through the oesophagus.

The super-absorbent particles are preferably produced using a sodium polyacrylate polymer. For example, super-absorbent particles sold under the trade name “Favor PAC 230” by the DEGUSSA company or those sold under the name “Norsocryl D60” by ATOFINA can be used.

To obtain the desired ballast effect, the mass of the super-absorbent particles inserted into the balloon 1 can be on the order of 1 g, for a balloon of around 600 cm³.

According to another alternative of the invention shown in FIG. 5, the intragastric balloon 1 includes a second flexible pouch 5, arranged so that it contains the first flexible pouch 2.

According to this alternative, the ballasting means 3 are contained in said second flexible pouch 5.

Thus, the volume containing the ballasting means 3 is defined by the wall 4 of the first flexible pouch 2, and by the surface of the second flexible pouch 5.

In this alternative embodiment, the impermeable coating, and, for example, the parylene, can be deposited on the surface of the first pouch 2 and/or on the surface of the second pouch 5.

As shown in FIG. 5, the ballasting means 3 used in this alternative embodiment can include super-absorbent particles.

Of course, the ballasting means can also comprise other absorbent bodies, such as foam, or more preferably solid dense bodies 3S such as those shown in FIG. 1.

According to an even more preferred embodiment of the invention, the intragastric balloon 1 comprises at least one sheath 8 capable of containing the ballasting means 3.

Such a sheath 8 thus makes it possible to prevent the ballasting means 3 from randomly dispersing within the intragastric balloon 1, and also enables, by containing the ballasting means 3, the introduction and passage of the intragastric balloon 1 through the patient's oral passageways to be facilitated.

In a particularly advantageous manner, the sheath 8 comprises two ends 8A, 8B, and is secured to the intragastric balloon 1 near at least one of said ends 8A, 8B.

The sheath 8 is preferably arranged, at least partially, inside the first pouch so that at least one of its two ends 8A, 8B is secured and preferably attached to a cap 6 for sealing the first pouch 2.

Of course, it is also possible to consider providing the intragastric balloon 1 according to the invention with an external sheath 8 (not shown in the Figs.), which would be located outside said first pouch 2.

However, to avoid the risks of perforation of the gastric wall or of infection, it is preferable to opt for the first solution, namely a sheath 8 contained inside the intragastric balloon 1.

According to an even more preferred alternative of the invention, the sheath 8 is secured to the intragastric balloon 1 at each of its ends 8A, 8B.

According to this alternative, the ends 8A, 8B of the sheath 8 are respectively secured to the valve 7 and the cap 6.

Such an assembly of the sheath 8 thus makes it possible to align the ballasting means 3 along the axis of implantation or extraction of the balloon, so as to facilitate the insertion or removal of the latter through the oesophagus (FIG. 3).

In a particularly advantageous manner, one of the ends 8A, 8B of the sheath 8 extends outside of the intragastric balloon 1, so as to form a pull tab 9.

Such a tab 9 thus has the advantage of facilitating the balloon extraction operation performed by the surgeon, in particular by enabling the latter to locate and grasp the balloon more easily.

Thus, if the surgeon pulls on the tab 9, the ballasting means 3 will line up along the sheath 8 so that the extraction of the balloon in the direction of extension of the sheath 8 will be facilitated.

The cap 6 can thus be arranged so as to allow one of the ends 8A, 8B of the sheath to pass through, so as to form the tab 9.

To ensure the impermeability of the balloon 1, the sheath 8 can be attached by adhesive to the cap 6 using, for example, a biocompatible adhesive or silicone or cyanoacrylate.

In a particularly advantageous manner, the material forming the tab 9 will preferably be selected so that it is resistant to the acidity of the gastric juices, and to the pulling stress that may be exerted by the surgeon during the balloon 1 extraction operation.

According to a preferred alternative of the invention, the sheath 8 can be deformed so as to avoid any rigidity that may, for example, lead to a perforation of the first pouch 2 of the balloon 1. However, this alternative is only preferred, and the use of a sheath 8 in the form of a rigid tube would not go beyond the scope of the invention.

To provide adequate flexibility and strength, the sheath 8 is preferably made of a biocompatible textile material or a material comprising silicone.

The sheath 8 is also preferably porous to liquids such as physiological liquid or water, so as to enable the absorbent bodies 3A optionally contained in the sheath to soak it up.

The sheath 8 can be made of a porous biocompatible textile mesh.

According to another alternative of the invention not shown in the Figs., the solid dense bodies 3S can be agglomerated in the sheath, in particular when the latter is made of an elastomer such as silicone.

Thus, the weights or solid bodies 3S will then preferably be embedded in an elastomer matrix, so as to substantially inhibit their relative mobility, while maintaining good flexibility of the sheath 8 and therefore of the intragastric balloon 1.

If the intragastric balloon 1 comprises a second flexible pouch 5, it is not necessary in principle to use a sheath 8 insofar as the concentric pouches 2, 5 define an interstitial space 10 as a sheath does.

According to a preferred alternative embodiment shown in FIG. 3, the sheath 8 comprises a plurality of compartments 8C in which the ballasting means 3 are distributed.

Such compartments 8C make it possible, by dividing the ballasting means 3, to better distribute the ballast in the balloon 1 and in particular to move the centre of gravity of the balloon 1 toward its geometric centre C, thus improving the overall mobility of the balloon (FIGS. 1 and 3).

In addition, such a distribution of the ballasting means 3 in a plurality of compartments 8C makes it possible to avoid shocks, in particular when the ballasting means 3 includes solid bodies 3S such as metal balls.

The compartments 8C are preferably aligned in the direction of extension of the sheath 8, so as to facilitate the implantation or extraction of the intragastric balloon.

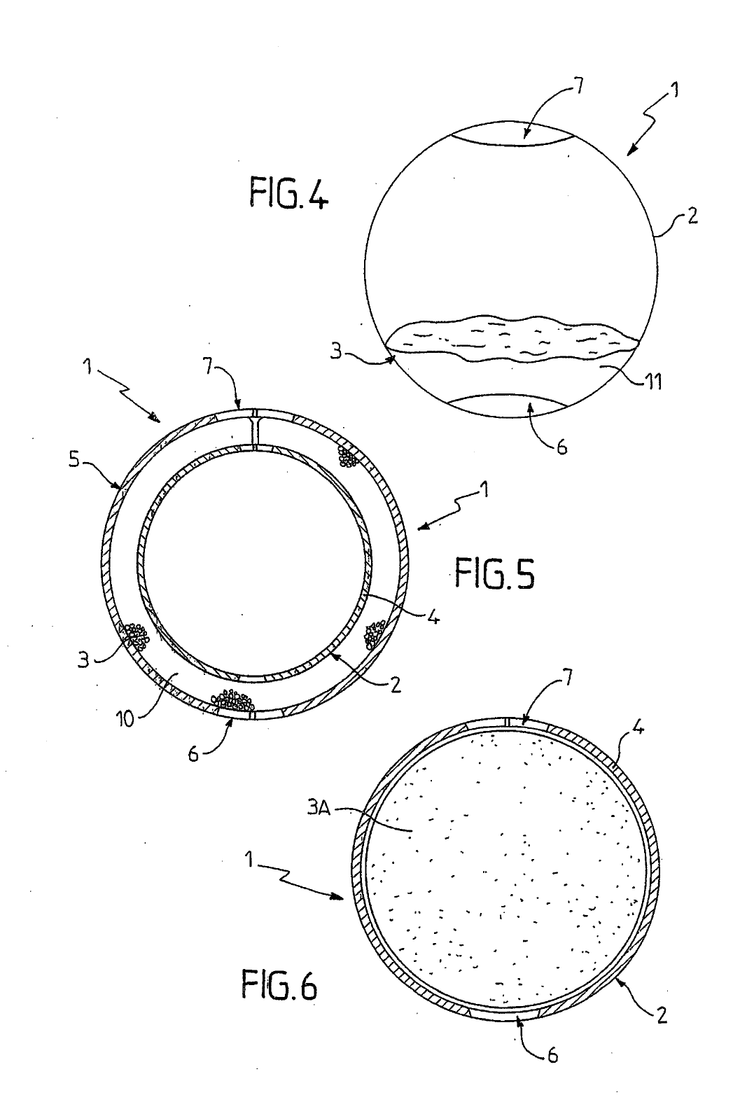

According to another alternative of the invention shown in FIG. 4, the ballasting means can include a liquid, intended to be introduced into the first pouch 2 to form a ballast.

The liquid 11 can either be used in combination with absorbent bodies 3A so as to be absorbed by the latter, or it can be used alone as a ballasting means 3.

In this embodiment, the inflation fluid is a gas, for example air, and the liquid is intended to be introduced by the surgeon into the first pouch 2 through the valve 7, so as to provide a ballasting function. However, the liquid 11 does not perform an balloon 1 inflation function, said function being provided by the inflation gas.

Thus, the use of an inflation gas is preferred to that of an inflation liquid so as to obtain a lighter balloon, capable of being better supported by the patient than a heavy balloon, such as a balloon entirely filled with water or a physiological liquid.

According to this alternative embodiment, the volume occupied by the ballasting means 3, namely the liquid 11, will preferably be at most on the order of 10% of the internal volume of the first pouch 2.

Therefore, the essential function of the liquid 11 will be to ballast the balloon 1, excluding any function of inflating said balloon.

Of course, it is also possible to consider producing intragastric balloons 1 already equipped with at least some of the ballasting means 3 in liquid form.

However, the first solution consisting of introducing the ballasting liquid 11 only after implantation of the balloon 1 in the stomach is preferably, insofar as it makes it possible to reduce the volume occupied by the balloon during the implantation step, and therefore to make the latter easier and faster.

The ballasted intragastric balloon 1 according to the invention therefore has properties enabling its positioning in the stomach to be improved while allowing it to be implanted easily and quickly through the oral passageway.

Another advantage of such a balloon is that it is particularly easy to implement and has a reduced production cost, while still ensuring the patient's safety.

Another advantage of the intragastric balloon 1 according to the invention comes from its actual structure, which gives it good mobility within the gastric cavity.

The invention also relates to a use of an absorbent body 3A to form a ballast for an expandable intragastric balloon 1.

The absorbent body 3A advantageously comprises a super-absorbent material capable of absorbing up to 200 times its weight in water.

The super-absorbent material preferably includes a sodium polyacrylate polymer.

According to an alternative of the invention, the absorbent body 3A can comprise a sponge or an absorbent foam. According to this alternative, the absorbent sponge will preferably have a very high compressibility ratio so as to limit the volume occupied by said sponge in the dry state, and to facilitate the implantation of the intragastric balloon 1.

The sponge is thus preferably made of a material comprising polyvinyl alcohol (PVA).

The invention also relates to a use of solid dense bodies 3S for forming a ballast for an expandable intragastric balloon 1. the solid dense bodies 3S preferably comprise tungsten.

The final placement of the intragastric balloon 1 according to the invention will now be described.

Before its implantation, the intragastric balloon 1 according to the invention is preferably in folded form, or even compressed so as to facilitate its insertion and passage through the patient's oral passageways, and in particular the oesophagus.

Once positioned inside the patient's stomach, the intragastric balloon 1 equipped with its ballasting means 3 must undergo a plurality of operations by the surgeon so as to be rendered functional, i.e. to give it a sufficient volume so that it will occupy a portion of the gastric cavity space reserved for food, and to render the ballasting means 3 operational.

Thus, if the ballasting means 3 comprise one or more solid dense bodies 3S, the surgeon can simply inflate the balloon, for example, using a gas-type inflation fluid, so as to cause it to change from its folded position to the expanded position.

However, if the ballasting means 3 include absorbent bodies 3A, and for example super-absorbent particles, such as water grains, the surgeon must, in addition to the step of inflating the balloon, perform a step in which he introduces a liquid, for example water or a physiological liquid, capable of being absorbed by the absorbent bodies 3S.

In particular, for a balloon having an internal volume of around 600 cm³, the surgeon can introduce around 60 cm³ of water to be soaked up by a predetermined mass of water grains located inside the balloon 1.

However, the intragastric balloon 1 according to the invention is structured so that the surgeon has the freedom to adapt the weight of the ballast to a given obesity treatment for a given patient.

The invention is industrially applicable in implantable devices for treating obesity.

1. An expandable intragastric balloon intended to be implanted inside the stomach of a patient so as to reduce the volume of the stomach in order to treat obesity, which balloon is equipped with at least one first flexible pocket capable of changing from a folded position to an expanded position by introducing an inflation fluid into the first pouch, wherein said expanded position gives the balloon its functional form, comprising means for ballasting said balloon, enabling the balloon to be substantially weighted so as to improve the positioning of the balloon in the stomach.

2. The intragastric balloon of claim 1, wherein the ballasting means are structurally integrated with the balloon.

3. The intragastric balloon of claim 1 wherein the ballasting means are located inside said first pouch.

4. The intragastric balloon of claims 1 wherein the ballasting means comprise at least one solid dense body capable of forming a ballast.

5. The intragastric balloon of claim 4, wherein the ballasting means comprise a plurality of solid dense bodies.

6. The intragastric balloon of claim 5, wherein the solid dense bodies are connected to one another so as to limit their relative mobility.

7. The intragastric balloon claim 6, wherein the ballasting means comprise spacers, arranged between two consecutive solid bodies so as to prevent shocks.

8. The intragastric balloon of claims 1 wherein the ballasting means include at least one absorbent body capable of forming a ballast in the presence of moisture.

9. The intragastric balloon of claim 8, wherein the absorbent body is formed by a sponge or a foam.

10. The intragastric balloon of claim 9, wherein the sponge is made of a polyvinyl alcohol-based material.

11. The intragastric balloon of claim 8, wherein the ballasting means include a plurality of absorbent bodies formed by super-absorbent particles of sodium polyacrylate polymer.

12. The intragastric balloon of claim 1 further comprising a second flexible pouch, said second flexible pouch being arranged so that it contains the first flexible pouch, with the ballasting means being contained in said second flexible pouch.

13. The intragastric balloon of claims 1 further comprising at least one sheath capable of containing the ballasting means.

14. The intragastric balloon of claim 13, wherein the sheath comprises two ends which sheath is secured to the balloon near at least one of said ends.

15. The intragastric balloon of claim 14, wherein one end of the sheath extends outside the balloon to form a pull tab.

16. The intragastric balloon of claim 13, wherein the sheath is deformable.

17. The intragastric balloon of claim 13, wherein the sheath is made of a biocompatible material.

18. The intragastric balloon of claim 1, wherein the surface of the first pouch is coated, with an impermeable parylene coating.

19. The intragastric balloon of claim 1 wherein the inflation fluid is a gas.

20. The intragastric balloon of claim 19, wherein the ballasting means include a liquid intended to be introduced into the first pouch so as to form a ballast.

21. The use of an absorbent body to form a ballast for an expandable intragastric balloon.

22. The use of claim 21, wherein the absorbent body comprises a super-absorbent material based on sodium polyacrylate.

23. The use of claim 21, wherein the absorbent body comprises a sponge or a foam.

24. The use of claim 23, wherein the material forming the sponge comprises polyvinyl alcohol.

25. The use of solid dense bodies as ballasting means for or in an expandable intragastric balloon.

26. The use according to claim 25, wherein the solid dense bodies comprise tungsten.

27. The intragastric balloon of claim 2, wherein the ballasting means are located inside said first pouch.

28. The intragastric balloon of claim 2, further comprising a second flexible pouch, said second flexible pouch being arranged so that it contains the first flexible pouch, with the ballasting means being contained in said second flexible pouch.
